(19) United States
(12) Patent Application Publication
(10) Pub. No.: US 2025/0009790 A1
(43) Pub. Date: Jan. 9, 2025

(54) METHODS FOR INDUCING VASODILATION

(71) Applicants: THE FLOREY INSTITUTE OF NEUROSCIENCE AND MENTAL HEALTH, Parkville, Victoria (AU); THE UNIVERSITY OF MELBOURNE, Parkville, Victoria (AU)

(72) Inventors: Scott AYTON, Parkville (AU); Ashenafi BETRIE, Parkville (AU); Ashley BUSH, Parkville (AU); Yugeesh LANKADEVA, Parkville (AU); Christine WRIGHT, Parkville (AU); James ANGUS, Parkville (AU)

(21) Appl. No.: 18/562,459

(22) PCT Filed: May 24, 2022
(86) PCT No.: PCT/AU2022/050494

§ 371 Date: Nov. 20, 2023

(30) Foreign Application Priority Data
May 24, 2021 (AU) 2021901532

(51) Int. Cl. A61K 33/30; A61K 9/00; A61P 9/08

(52) U.S. Cl. CPC A61K 33/30; A61K 9/0019; A61P 9/08

(57) ABSTRACT

Provided herein are methods for preventing or minimising hypoperfusion and hypoxia during surgery, methods for preventing, or reducing the risk of, post-operative complications associated with hypoperfusion and/or hypoxia and methods for treating and preventing vascular diseases and disorders, the methods comprising administering to subjects in need thereof, a zinc ion and at least one amino acid or a salt thereof.

### FIELD

The present invention relates to the use of zinc delivery agents comprising a zinc ion and at least one amino acid or a salt thereof for inducing vasodilation. More particularly, the present invention relates to methods for preventing or minimising hypoperfusion and hypoxia during surgery, methods for preventing, or reducing the risk of, post-operative complications associated with hypoperfusion and/or hypoxia and methods for treating and preventing vascular diseases and disorders by administering zinc delivery agents described herein.

### BACKGROUND

Vascular diseases and disorders may arise from abnormal vascular tone regulation. Vascular tone refers to the contractile activity of vascular smooth muscle cells in the walls of blood vessels. Since the vasculature extends throughout the human body, diseases and disorders associated with abnormal vascular tone can have wide-ranging effects on various organs and tissues, both centrally and peripherally.

Some vascular disorders affect cutaneous blood flow, in contrast to systemic blood vessels. Raynaud's phenomenon is a disorder characterized by chronic ischaemic attacks to arteries supplying fingers and other extremities, which leads to reduced cutaneous blood flow, pain and ulcers. An example of other disorders related to reduced cutaneous blood flow includes gangrene. There are currently no approved treatment options for the management of Raynaud's phenomenon. Antihypertensive vasodilatory drugs may be prescribed, however these are only used for management of the symptoms (and not the disorder itself) and are associated with unwanted side effects including hypotension.

Metal-based drugs for the treatment of cardio- and cerebrovascular diseases typically target cellular processes that modify calcium or potassium (i.e. s-block elements), however these drugs often have unwanted side effects or may have limited efficacy. The role that other metal types (for example, transition metals) may play in cardiovascular physiology has not, to date, led to the development of drugs that are useful in clinical settings.

There remains a need for new agents for the treatment of vascular diseases and disorders associated with reduced cutaneous blood flow that do not induce unwanted side effects.

Reduced blood flow during surgery is also increasingly being recognized as a critical factor in a number of post-operative complications. In particular there is emerging evidence that intra-operative hypoperfusion (reduced blood flow) and hypoxia (reduced oxygenation) within the brain and neuroinflammation (activated microglia) are critical drivers of delirium after cardiopulmonary bypass.

Approximately 2 million cardiac surgical procedures requiring cardiopulmonary bypass are performed annually worldwide. Major unresolved complications arising from cardiopulmonary bypass include post-operative delirium that occurs in ˜50% of patients (about 20% of which develop long-term cognitive decline) and acute kidney injury that occurs in 20-30% of patients. These patients are at high risk of developing chronic kidney disease. Cardiac surgery-associated postoperative complications, particularly those arising from injury to the brain and kidneys, continues to significantly increase morbidity and mortality in patients. Evidence suggests that renal medullary hypoperfusion and hypoxia are common pathophysiological features of acute kidney injury arising from cardiopulmonary bypass and also in the progression of acute kidney injury to chronic kidney disease (Evans et al., 2018, Acta Physiol 222, e12995).

There are currently no interventions to prevent or treat cardiopulmonary bypass-associated delirium and acute kidney injury.

### SUMMARY OF THE DISCLOSURE

According to one aspect, the present invention provides a method for preventing or minimising hypoperfusion in a subject during surgery, the method comprising administering to the subject a therapeutically effective amount of a zinc delivery agent, wherein the zinc delivery agent comprises a zinc ion and at least one amino acid or a salt thereof.

Typically, the administration of the zinc delivery agent results in an improvement in one or more of cerebral cortical perfusion, renal cortical perfusion and renal medullary perfusion during the surgery. Typically, in accordance with the above aspects the administration of the zinc delivery agent results in an improvement in one or more of renal blood flow, vascular resistance and renal oxygen delivery during the surgery.

Typically, the administration of the zinc delivery agent does not result in a decrease in systemic mean arterial pressure. Typically, in accordance with the above aspects the administration of the zinc delivery agent maintains a systemic mean arterial pressure of at least about 50 mmHg, preferably at least about 65 mmHg.

The zinc delivery agent may be administered prior to surgery, during surgery and/or after surgery. In an embodiment, the zinc delivery agent is administered intravenously.

In an exemplary embodiment, the surgery is vascular surgery, abdominal surgery, orthopaedic surgery or transplant surgery. In a particular exemplary embodiment the surgery is cardiac surgery requiring cardiopulmonary bypass.

In an embodiment, the method prevents, or reduces the risk of, the subject suffering from post-operative complications associated with reduced blood flow and reduced oxygen delivery to tissues, typically neurological and renal complications. Exemplary post-operative neurological complications include delirium. Exemplary post-operative renal complications include acute kidney injury and subsequent chronic kidney disease.

Accordingly, another aspect of the invention provides a method for preventing, or reducing the risk of, post-operative complications associated with hypoperfusion and/or hypoxia in a subject, the method comprising administering to the subject a therapeutically effective amount of a zinc delivery agent, wherein the zinc delivery agent comprises a zinc ion and at least one amino acid or a salt thereof.

In another aspect, the present invention provides a method for the treatment or prevention of a vascular disease or disorder in a subject, the method comprising administering to the subject a therapeutically effective amount of a zinc delivery agent, wherein the zinc delivery agent comprises a zinc ion and at least one amino acid or a salt thereof.

In an exemplary embodiment, the vascular disease or disorder is Raynaud's phenomenon. In a particular embodiment, Raynaud's phenomenon is primary Raynaud's phenomenon or secondary Raynaud's phenomenon.

In another aspect, the present invention provides a method for the treatment or prevention of an autoimmune disease associated with Raynaud's phenomenon, the method comprising administering to a subject in need thereof a therapeutically effective amount of a zinc delivery agent, wherein the zinc delivery agent comprises a zinc ion and at least one amino acid or a salt thereof.

In an embodiment, the autoimmune disease associated with Raynaud's phenomenon is systemic sclerosis, a mixed connective tissue disease, systemic lupus erythematosus, primary Sjogren's syndrome, a myositis-spectrum disorder or fibromyalgia syndrome.

In another aspect, the present invention provides a method for the modulation of vascular tone in a subject, the method comprising administering to the subject a therapeutically effective amount of a zinc delivery agent, wherein the zinc delivery agent comprises a zinc ion and at least one amino acid or a salt thereof.

In an embodiment, the modulation of vascular tone results in vasodilation. The present inventors have found that the vasodilation observed occurs without the natural drop in systemic blood pressure that results from a normal widening of a blood vessel.

Typically, the vasodilation occurs in blood vessels highly innervated with sensory nerves. In an embodiment, the blood vessels are cutaneous blood vessels. Typically, the vasodilation in cutaneous blood vessels leads to an increase in cutaneous blood flow.

Also provided herein is the use of a zinc delivery agent in the manufacture of a medicament for preventing or minimising hypoperfusion during surgery, wherein the zinc delivery agent comprises a zinc ion and at least one amino acid or a salt thereof.

Also provided herein is the use of a zinc delivery agent in the manufacture of a medicament for preventing, or minimizing the risk of, post-operative complications associated with hypoperfusion and/or hypoxia, wherein the zinc delivery agent comprises a zinc ion and at least one amino acid or a salt thereof.

Also provided herein is the use of a zinc delivery agent in the manufacture of a medicament for the treatment or prevention of a vascular disease or disorder, wherein the zinc delivery agent comprises a zinc ion and at least one amino acid or a salt thereof.

Also provided herein is the use of a zinc delivery agent in the manufacture of a medicament for the treatment or prevention of an autoimmune disease associated with Raynaud's phenomenon, wherein the zinc delivery agent comprises a zinc ion and at least one amino acid or a salt thereof.

Also provided herein is the use of a zinc delivery agent in the manufacture of a medicament for the mediation of vascular tone, wherein the zinc delivery agent comprises a zinc ion and at least one amino acid or a salt thereof.

In accordance with the aspects and embodiments hereinbefore described, in an exemplary embodiment the amino acid or salt thereof is histidine or a histidine salt. Optionally, the zinc ion and the at least one amino acid, or salt thereof, are provided in the form of a complex, or form a complex in vivo after administration to the subject.

In accordance with the aspects and embodiments hereinbefore described, in an exemplary embodiment the delivery agent comprises zinc bis(histidinate).

### BRIEF DESCRIPTION OF THE FIGURES

Embodiments of the invention are described herein, by way of non-limiting example only, with reference to the following figures.

FIG. 1. Relaxation responses to various zinc delivery agents in rat mesenteric arteries contracted with the thromboxane-mimetic, U46619 (U tone). Relaxation was found to be dependent on concentration of the zinc delivery agent. Administration of zinc alone caused little relaxation.

FIG. 2. Relaxation responses to administration of zinc bis(histidinate) with different stereoisomers of histidine or a stereoisomer of histidine alone in rat mesenteric arteries contracted with the thromboxane mimetic, U46619 (U tone). Administration of histidine alone caused little relaxation. Relaxation was found to be dependent on the concentration of the zinc-bis-histidinate administered.

FIG. 3. Laser Doppler flux of rat hind paw after administration of zinc bis(histidinate) (Zn-bis-His; 3, 10 and 30 mg/kg i.v.) and histidine alone (L-His; 67 mg/kg i.v.). Zinc bis(histidinate) caused an increase in cutaneous blood flow; histidine alone did not cause an increase in cutaneous blood flow.

FIG. 4. Mean arterial pressure after zinc bis(histidinate) bolus intravenous injection. (Zn-bis-His) or equivalent dose of L-histidine (L-His, 67 mg/kg) for the highest dose of Zn-bis-His tested. Zinc bis(histidinate) does not change mean arterial pressure. Error bars are t SEM. n, number of rats; all 7 rats studied were used to test all the 3 doses of Zn-bis-his at the time indicated, but only 5 received the subsequent dose of L-His.

FIG. 5. Area under the vasodilation curve with and without BIBN4096 treatment in vivo. Zinc bis(histidinate) (Zn-bis-His; 3, 10 and 30 mg/kg i.v.) caused an increase in cutaneous blood flow, which was dependent on sensory nerve activity, since the inhibition of CGRP receptors with BIBN4096 pretreatment (at a dose that blocks sensory nerve stimulation-induced vasodilatation, 3 mg/kg i.v.) attenuated the effect.

FIG. 6. Decreasing plasma zinc levels (μmol/L) in a cohort of human patients that underwent cardiopulmonary bypass (n=82) at different time points during bypass surgery (on bypass and off bypass) and then hours post-surgery (ICU, intensive care unit). Zinc levels were analysed by ICP-MS and data expressed as mean mean±standard deviation. **** p<0.00001, compared to the “1-Induction” value, repeated measured one-way ANOVA with Dunnett's post-test. ICU, intensive care unit.

FIG. 7. Zinc deficiency in (A) humans (n=82) and (B) sheep (n=14) 2-hours after starting cardiopulmonary bypass (CPB). **** p<0.00001, paired Student t-test.

FIG. 8. Effects of cardiopulmonary bypass (CPB) (2 h) and increasing dose of zinc bis(histidinate) (ZBH) at 3, 10 and 30 mg/kg infusion i.v. every 20 min after CPB on mean arterial blood pressure (A), renal blood flow (B), renal vascular resistance (C) and renal oxygen delivery (D) in two sheep (filled circles and filled squares). The time-control effects of CPB on 6 control sheep are shown by filled triangles (expressed as mean±standard deviation).

FIG. 9. Effects of cardiopulmonary bypass (CPB) (2 h) and increasing dose of zinc bis(histidinate) (ZBH) at 3, 10 and 30 mg/kg infusion i.v. every 20 min after CPB on cerebral cortical (A), renal cortical (B) and renal medullary (C) blood perfusion in two sheep (filled circles and filled squares). The time-control effects of CPB on 6 control sheep are shown by filled triangles (expressed as mean±standard deviation).

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

The articles “a” and “an” are used herein to refer to one or to more than one (i.e., to at least one) of the grammatical object of the article. By way of example, “an element” means one element or more than one element.

In the context of this specification, the term “about” is understood to refer to a range of numbers that a person of skill in the art would consider equivalent to the recited value in the context of achieving the same function or result.

Throughout this specification and the claims which follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

The term “optionally” is used herein to mean that the subsequently described feature may or may not be present or that the subsequently described event or circumstance may or may not occur. Hence the specification will be understood to include and encompass embodiments in which the feature is present and embodiments in which the feature is not present, and embodiments in which the event or circumstance occurs as well as embodiments in which it does not.

The term “inhibitor” as used herein refers to an agent that decreases or inhibits at least one function or biological activity of a target molecule or receptor.

In the context of the present disclosure, the terms “inhibiting” and grammatical equivalents do not necessarily imply the complete inhibition of the specified event, activity or function. Rather, the inhibition may be to an extent, and/or for a time, sufficient to produce the desired effect. Inhibition may be prevention, retardation, reduction or otherwise hindrance of the event, activity or function. Such inhibition may be in magnitude and/or be temporal in nature. In particular contexts, the terms “inhibit” and “prevent”, and variations thereof may be used interchangeably. Similarly, the terms “inhibit”, “decrease” and “reduce” may be used interchangeably, in reference to the level of, or a value for, a substance, phenomenon, function or activity in a second sample or at a second timepoint that is lower than the level of, or value for, the substance, phenomenon, function or activity in a first sample or at a first timepoint. The reduction may be determined or measured subjectively or objectively, and may be subject to an art-accepted statistical method of analysis.

As used herein the terms “treating”, “treatment”, “preventing”, “prevention” and grammatical equivalents refer to any and all uses which remedy the stated neurodegenerative disease, prevent, retard or delay the establishment of the disease, or otherwise prevent, hinder, retard, or reverse the progression of the disease. Thus the terms “treating” and “preventing” and the like are to be considered in their broadest context. For example, treatment does not necessarily imply that a patient is treated until total recovery. Where the disease displays or a characterized by multiple symptoms, the treatment or prevention need not necessarily remedy, prevent, hinder, retard, or reverse all of said symptoms, but may prevent, hinder, retard, or reverse one or more of said symptoms.

The term “subject” as used herein refers to mammals and includes humans, primates, livestock animals (e.g. sheep, pigs, cattle, horses, donkeys), laboratory test animals (e.g. mice, rabbits, rats, guinea pigs), performance and show animals (e.g. horses, livestock, dogs, cats), companion animals (e.g. dogs, cats) and captive wild animals. Preferably, the mammal is human or a laboratory test animal. Even more preferably, the mammal is a human.

Zinc is an abundant transition metal and is essential for many proteins that serve structural, functional and signaling functions in cardiovascular biology. Proteins such as nitric oxide synthase, phosphodiesterase, angiotensin converting enzyme, superoxide dismutase, neprilysin and angiotensin II either directly bind to zinc or depend on the presence of zinc for activity. It has been shown that zinc supplements decrease systolic blood pressure, while a deficiency of zinc is associated with high blood pressure. Cellular concentrations of zinc are controlled by 24 zinc transporter channels that have varied expression in different organs and cells.

The present invention is predicated on the findings that vascular tone can be mediated by the action of zinc that is delivered by a suitable delivery agent. The present inventors have found that intracellular zinc plays a fundamental role in maintaining vascular health by mediating vasorelaxation within vital organs including the brain and kidneys. As shown herein, the inventors have identified that the administration of a zinc delivery agent comprising a zinc ion and at least one amino acid or a salt thereof leads to an increase in the cytoplasmic concentration of zinc and subsequently relaxation of vascular smooth muscle. Without wishing to be bound by theory, the present inventors have found that zinc-mediated vasodilation can occur by at least three different cellular mechanisms and is a result of the presence of zinc in the cytoplasm. The present disclosure relates to the administration of a zinc ion and at least one amino acid or salt thereof, optionally a complex of said zinc ion and at least one amino acid or salt thereof, that leads to an increase in the cytoplasmic concentrations of zinc in vascular tissue.

The present inventors have found that the administration of zinc to increase cytoplasmic concentrations of the metal in vascular tissue leads to vasodilation resulting from smooth muscle relaxation. Relaxation of vascular smooth muscle is due to the zinc-mediated activation of the transient receptor potential cation channel subfamily A (ankyrin) member 1 (TRPA1) channel, zinc-mediated inhibition of voltage-gated calcium channels (VGCC) and the zinc-mediated increase in dilatory prostanoid signaling. Each of these three mechanisms contribute to the observed vasodilation when cytoplasmic concentrations of zinc are increased.

The present inventors have surprisingly found that delivering zinc across the cell membrane by coordinating a zinc ion with a ligand that may be transported by other means, leads to an increase in cytoplasmic zinc concentrations. This means that traditional zinc transporters can be bypassed.

Accordingly, provided herein is a method for the modulation of vascular tone in a subject, comprising administering to the subject a therapeutically effective amount of a zinc delivery agent, wherein the zinc delivery agent comprises a zinc ion and at least one amino acid or a salt thereof. The modulation of vascular tone in accordance with the present invention results in vasodilation.

The present inventors have found that the vasodilation observed using the zinc delivery agents disclosed herein occurs without the reduction in systemic blood pressure that normally results from a widening of a blood vessel. Without wishing to be bound by theory, the present inventors suggest that this is due to the zinc delivery agent not exerting a therapeutic effect on arteries responsible for control of blood pressure in vivo. Alternatively, the administration of the zinc delivery agent may result in the activation of mechanisms to compensate for a reduction in blood pressure owing to the zinc-mediated vasodilation, such that no appreciable decrease in blood pressure is observed.

As exemplified herein, the inventors have shown that the vasodilation in cutaneous blood vessels caused by zinc bis(histidinate) leads to increased cutaneous blood flow, and that this is dependent on sensory nerve activity; as shown herein the inhibition of CGRP (calcitonin gene-related peptide) receptors prior to zinc bis(histidinate) administration reduced cutaneous blood flow. Without wishing to be bound by theory, the inventors propose that administration of zinc delivery agents of the present disclosure leads to the release of CGRP from sensory nerves, thus producing a significant vasodilatory effect on blood vessels highly innervated with CGRP-containing sensory nerves. Cutaneous blood vessels are highly innervated with sensory nerves, as are, for example, renal blood vessels.

Also provided herein is a method for the treatment or prevention of a vascular disease or disorder in a subject, the method comprising administering to the subject a therapeutically effective amount of a zinc delivery agent, wherein the zinc delivery agent comprises a zinc ion and at least one amino acid or a salt thereof.

As used herein, the term “vascular disease or disorder” refers to disease or disorder that affects the vasculature or blood vessels, i.e. the arteries, veins and/or capillaries, of a physiological system and the flow of blood through these vessels. The flow of blood through the vessels may be blocked or reduced as a result of damage to, decreased diameter of or weakening of blood vessels. Reduced or absent blood flow may then adversely affect organs or parts of the body associated with the vessels that are blocked. A subset of vascular disease includes ischaemic diseases, where there is decreased blood flow to a tissue, organ or part of the body. The decrease in blood flow may be attributed to various causes, for example, the build-up of plaque or fatty deposits in the wall of the blood vessels, which in turn reduces blood flow. Treatment of ischaemic disease ideally results in vasodilation and restoration of blood flow. Without wishing to be bound by theory, the present inventors believe that ischaemic disease may be treated by a zinc delivery agent as disclosed herein, by mediating vasodilation.

Examples of vascular diseases and disorders that may be treated by a zinc delivery agent as disclosed herein include Raynaud's phenomenon, vascular disease in diabetes, gangrene, intraoperative vasoconstriction, cutaneous constriction in response to vasopressor therapy, vascular stiffness, pulmonary hypertension, ischaemic stroke, vasospasm after subarachnoid haemorrhage, coronary artery spasm or vasospastic angina, hypoxia in organs after surgery and cerebrovascular dysfunction. The skilled person will appreciate that the scope of the present invention is not limited by reference to any one of these exemplary conditions.

As used herein, the term “Raynaud's phenomenon” refers to the manifestation of chronic ischaemic attacks to arteries and/or capillaries that restrict blood flow to the fingers and/or other extremities, such as the toes, ears or nose leading to colour changes in the skin owing to narrowing of the arteries. Narrowing of the arteries results from an imbalance between the constrictor and dilator arms of an artery, with excess vasoconstriction leading to Raynaud's phenomenon. Since narrowing of the arteries restricts blood flow, deoxygenation of the surrounding tissue also occurs. Since Raynaud's phenomenon is characterized by reduced cutaneous blood flow, administration of a zinc delivery agent as disclosed herein may be used to mediate vasodilation (and subsequently, cutaneous blood flow) and therefore treat or alleviate Raynaud's phenomenon. In contrast to other oral vasodilators such as calcium channel blockers often used in the management of Raynaud's phenomenon, administration of the zinc delivery agent comprising a histidine ligand does not lead to an unwanted decrease in mean arterial blood pressure.

Raynaud's phenomenon may be classified as either primary or secondary. Primary Raynaud's phenomenon (i.e. Raynaud's disease) occurs in the absence of an associated disease or disorder, while secondary Raynaud's phenomenon (i.e. Raynaud's syndrome) is associated with a known disease. Secondary Raynaud's phenomenon is often associated with autoimmune rheumatic diseases, for example, systemic sclerosis and mixed connective tissue diseases, systemic lupus erythematosus, primary Sjogren's syndrome, myositis-spectrum disorders and fibromyalgia syndrome.

Also provided herein is the treatment or prevention of diseases and disorders associated with Raynaud's phenomenon. For example, Raynaud's phenomenon is often associated with autoimmune diseases, and embodiments of the present invention therefore provide methods for the treatment or prevention of autoimmune diseases associated with Raynaud's phenomenon. The autoimmune disease associated with Raynaud's phenomenon may be, for example, systemic sclerosis, a mixed connective tissue disease, systemic lupus erythematosus, primary Sjogren's syndrome, a myositis-spectrum disorder or fibromyalgia syndrome.

As used herein, the term “therapeutically effective amount” refers to an amount sufficient to effect a beneficial or desired result. An effective amount is typically sufficient to palliate, ameliorate, stabilize, reverse, slow or delay the progression of the disease or disorder to be treated. An effective amount may be administered in one or more administrations. The exact amount required will vary from subject to subject and the nature of the disease or disorder. A “therapeutically effective amount” may be determined by one of ordinary skill in the art using only routine experimentation.

As exemplified herein the present inventors have found that when administered during cardiopulmonary bypass surgery, a zinc delivery agent of the present invention elevates intracellular zinc levels and results in a dose-dependent improvement in cerebral cortical, renal cortical and renal medullary microcirculatory perfusion and correspondingly in renal blood flow, vascular resistance and renal oxygen delivery. Significantly, these improvements are observed without compromising systemic mean arterial pressure.

As used herein, the term “improvement” means a beneficial change in a parameter, factor or physiological measure resulting from, or associated with, the administration of the zinc delivery agent compared to the absence of the agent. That is, an “improvement” may be an increase in the value of a parameter, factor or physiological measure in which an increase is desirable, or a decrease in the value of a parameter, factor or physiological measure in which a decrease is desirable, resulting from, or associated with, the administration of the zinc delivery agent compared to the value observed in the absence of the agent.

Accordingly, the present invention provides a method for preventing or reducing hypoperfusion in a subject during surgery, the method comprising administering to the subject a therapeutically effective amount of a zinc delivery agent, wherein the zinc delivery agent comprises a zinc ion and at least one amino acid or a salt thereof. The present invention also provides a method for preventing or reducing hypoxia in a subject during surgery, the method comprising administering to the subject a therapeutically effective amount of a zinc delivery agent, wherein the zinc delivery agent comprises a zinc ion and at least one amino acid or a salt thereof.

Broadly, hypoperfusion refers to a decrease or reduction in blood flow through a blood vessel. More specifically, hypoperfusion can refer to a level of perfusion, globally (throughout the body) or regionally (in one or more specific tissues) or even within the same organ, below that required for metabolic processes at a particular point in time. During major surgery there is a risk of hemodynamic disturbance and global or regional (e.g. cerebral or renal) hypoperfusion. Pre-operative preparations, surgical bleeding, and the effects of anaesthesia can increase the risk of changes in vascular resistance that can trigger arterial hypotension and global or regional hypoperfusion and ischaemia, both during and after surgery. Without being limiting on the scope of the present disclosure, in surgical settings a reduction in blood flow of at least about 30% (for example between about 30% and about 50%), compared to blood flow prior to the surgery, anaesthesia or pre-operative preparations, may be considered hypoperfusion. Perfusion can be determined or measured using any means known to those skilled in the art, including for example fluorescence analysis, laser Doppler perfusion probes/imaging, dynamic light scattering (or laser speckle contrast imaging, insertion of a thermal diffusion probe, imaging photoplethysmography, superb microvascular imaging, or renal medullary perfusion/oxygenation as indirectly assessed via measurement of bladder urinary oxygenation. The scope of the present disclosure is not limited by reference to any one means of determining or measuring hypoperfusion or any specific level of reduction of blood flow.

By preventing or minimising hypoperfusion during surgery, the methods described herein assist in avoiding post-operative complications resulting from, or associated with, reduced blood flow and/or reduced oxygen delivery to tissues during surgery. Such complications include, for example, cardiac, cognitive/neurological, renal and hepatic complications. An exemplary cognitive or neurological complication is post-operative delirium, which can lead to increased risk of developing dementia. An exemplary renal complication is post-operative acute kidney injury, which may result in chronic kidney disease. An exemplary hepatic complication is post-operative liver dysfunction, which can result in chronic liver disease.

Use of the term “associated with” herein describes a direct or indirect relationship between the occurrence of an event with an action or measure. Thus, a post-operative complication associated with surgery or with reduced blood flow or reduced oxygen delivery to tissue during surgery, means that the post-operative complication results from the surgery either directly or indirectly. The post-operative complication may occur or begin hours, days, weeks, months or years after the surgery. Thus, symptoms of a post-operative complication may not be apparent until hours, days, weeks, months or years after a surgery.

Systemic blood flow may be increased upon administration of the zinc delivery agent relative to the level of blood flow observed in the absence of the agent. Similarly, oxygen delivery to tissues, such as the kidneys, may be increased upon administration of the zinc delivery agent relative to the level of blood flow observed in the absence of the agent. Thus, hypoperfusion (reduced blood flow) or hypoxia (reduced oxygen delivery to tissues), may be prevented during surgery using the methods described herein. Alternatively, hypoperfusion or hypoxia may be minimized, i.e. reduced such that the level of any reduced blood flow or reduced oxygen delivery is insignificant and has no bearing on progress or outcome of the surgery. Thus, post-operative complications may be prevented, or the risk of a subject developing a post-operative complication may be reduced, whether the hypoperfusion or hypoxia is prevented or minimised.

Typically the administration of the zinc delivery agent does not result in a decrease in systemic mean arterial pressure during and after the surgery. Typically the administration of the zinc delivery agent maintains a systemic mean arterial pressure during the surgery of at least about 50 mmHg. For example, the administration of the zinc delivery agent may maintain a systemic mean arterial pressure during the surgery of between about 50 mmHg and about 80 mmHg, such as at least or about 50 mmHg, at least or about 55 mmHg, at least or about 60 mmHg, at least or about 65 mmHg, at least or about 70 mmHg, at least or about 75 mmHg, or at least or about 80 mmHg. The system mean arterial pressure may depend on the type of surgery.

By preventing or minimising hypoperfusion during surgery with no observable or significant decrease in systemic mean arterial pressure, the methods described herein can reduce or eliminate the need for vasopressors, such as metaraminol, which are otherwise commonly employed to maintain a target mean arterial pressure during surgery.

The zinc delivery agent may be administered to the subject one or more times, prior to surgery, during surgery and/or after surgery, typically intravenously. The number of administrations, timing of administrations and duration of administration can be readily determined by the skilled person, depending on a number of factors including the patient's condition, the type of surgery, the duration of surgery, the progress of the surgery and the patient's blood zinc levels at one or more time points before, during and after surgery. Pre-operative administration of the zinc delivery agent may comprise one or more administrations of the agent up to about 24 hours prior to surgery, for example about 24 hours, about 18 hours, about 12 hours, about 6 hours, about 2 hours, about 1 hour, about 30 mins or about 15 mins before surgery. Intra-operative administration of the zinc delivery agent may comprise one or more administrations of the agent as needed during the surgery and via a continuous intravenous infusion during the surgical procedure. Post-operative administration of the zinc delivery agent may comprise one or more administrations of the agent or a continuous intravenous infusion up to about 48 hours after the surgery, for example up to about 2 hours, about 6 hours, about 12 hours, about 24 hours or about 48 hours after the surgery. Multiple administrations of the zinc delivery agent may be desired, and/or the agent may be administered to the subject such that the desired dose is delivered over a suitable period of time peri-operatively, for example over a period of between about 30 mins to several hours, depending on factors such as the patient's condition, the type of surgery, the duration of surgery and the progress of the surgery. The timing and number of administrations may be determined, for example, based on zinc levels in the blood of the patient, wherein a reduced blood zinc concentration relative to the concentration in the patient prior to surgery or prior to the pre-operative preparation or anaesthesia indicates the continued need for administration of the zinc delivery agent.

In embodiments, as exemplified herein, in which the zinc delivery agent comprises zinc bis(histidinate), a dose (individual administration) of the agent may comprise from about 0.1 mg/kg to about 1000 mg/kg zinc bis(histidinate), or from about 0.1 mg/kg to about 500 mg/kg zinc bis(histidinate), or from about 1 mg/kg to about 100 mg/kg zinc bis(histidinate), or from about 1 mg/kg to about 50 mg/kg zinc bis(histidinate), for example about 3 mg/kg, about 10 mg/kg, or about 30 mg/kg zinc bis(histidinate). The amount or concentration to be administered can be readily determined by the skilled person based on a number of factors such as the patient's condition, the type of surgery, the duration of surgery, the progress of the surgery and the patient's blood zinc levels at one or more time points before, during and after surgery.

Surgeries in which the methods described above can be employed are any surgery in which hypoperfusion may be a risk, for example any surgery requiring administration of a general anaesthetic. The surgery may be, for example, vascular surgery, abdominal surgery, transplant surgery, or orthopaedic surgery, such as joint replacement surgery. The vascular surgery may be, for example, cardiac surgery. The transplant surgery may be, for example, heart, liver or lung transplant surgery. The orthopaedic surgery may be, for example, joint replacement surgery. In an exemplary embodiment, the surgery is cardiac surgery requiring cardiopulmonary bypass.

The inventors have found that the therapeutic effects observed and described herein occur when both zinc and at least one amino acid are administered together as the zinc delivery agent. In some embodiments, the zinc delivery agent contains zinc and an amino acid in about a 1:1 ratio. In another embodiment, the zinc delivery agent contains zinc and an amino acid in about a 1:2 ratio.

The composition and structure of the zinc delivery agent as discussed herein may depend on both the oxidation state of the zinc ion and the at least one amino acid. For example, the delivery agent comprising a zinc ion and at least one amino acid may be a two-, four- or six-coordinate metal complex, i.e. where the amino acid has multiple bonds or interactions with the zinc ion. Where a metal complex between the zinc ion and the amino acid is formed, the bonds between the zinc ion and amino acid may be covalent coordination bonds between an appropriate atom of the amino acid and the zinc ion. In some instances, the metal complex between the zinc ion and the amino acid may include other components coordinated to the zinc ion, for example, water or other suitable ligands.

In accordance with embodiments of the present invention, the zinc ion and the at least one amino acid or salt thereof may be provided in the form of a complex, or a complex comprising the zinc ion and the at least one amino acid or salt thereof may form in vivo after administration of the delivery agent comprising the zinc ion and at least one amino acid or salt thereof to a subject.

As used herein, the term “amino acid” refers to a molecule which contains both an amino and a carboxyl functional group. The amino acid may be a natural or unnatural amino and may also be in equilibrium with its zwitterionic form. The amino acid may contain modifications at either the amino and/or carboxyl terminus, or may contain a free amino group or carboxyl group. Further modification of the amino acid side chain or additional substitutions at other parts of the amino acid are also contemplated.

As used herein, naturally occurring amino acids are the L- or D-form of the twenty amino acids commonly found in nature. These are glycine (Gly, G), alanine (Ala, A), valine (Val, V), leucine (Leu, L), isoleucine (Ile, I), methionine (Met, M), proline (Pro, P), phenylalanine (Phe, F), tryptophan (Trp, W), serine (Ser, S), threonine (Thr, T), asparagine (Asn, N), glutamine (Gln, Q), tyrosine (Tyr, Y), cysteine (Cys, C), lysine (Lys, K), arginine (Arg, R), histidine (His, H), aspartic acid (Asp, D), and glutamic acid (Glu, E).

As used herein, non-naturally occurring amino acids include any compound with both amino and carboxyl functionality, derivatives thereof, or derivatives of a naturally occurring amino acid. These amino acids can form part of a peptide chain through bonding via their amino and carboxyl groups. Alternatively, these derivatives may bond with other natural or non-naturally occurring amino acids to form a non-peptidyl linkage.

Non-naturally occurring amino acids may include amino acids that have undergone side chain modifications. Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

In accordance with exemplary embodiments of the present invention, the amino acid or amino salt is histidine or a histidine salt. In a particular exemplary embodiment, the delivery agent comprises histidine. In another embodiment, the amino acid is DL-histidine. In another embodiment, the amino acid is D-histidine. In another embodiment, the amino acid is L-histidine.

In particular exemplary embodiments of the present invention, the delivery agent comprises zinc bis(histidinate).

The present inventors have found that the ratio of the amino acid and the configuration of the amino acid in the zinc delivery agent may affect the extent of vasodilation observed. In an embodiment, the zinc delivery agent comprises or consists of a zinc ion and D-histidine. In another embodiment, the zinc delivery agent comprises or consists of a zinc ion and L-histidine. In a further embodiment, the zinc delivery agent comprises or consists of a zinc ion and D-histidine in a ratio of about 1:2. In another embodiment, the zinc delivery agent comprises or consists of a zinc ion and L-histidine in a ratio of about 1:2.

In embodiments of the present invention the zinc delivery agent comprises a zinc ion and a histidine ligand. In another embodiment, the zinc delivery agent comprises a zinc ion and more than one histidine ligand. In another embodiment, the zinc delivery agent comprises a zinc ion and two histidine ligands. In another embodiment, the zinc delivery agent comprises a zinc ion and four histidine ligands.

Optionally, the zinc ion and the histidine ligand are provided in the form of a complex. Alternatively, a complex comprising the zinc ion and the histidine ligand may form in vivo after administration of the delivery agent comprising the zinc ion and the histidine ligand to the subject.

Embodiments of the present disclosure contemplate the administration of a zinc delivery agent as described herein to subjects in need by any suitable means, and typically in the form of pharmaceutical compositions, which compositions may comprise one or more pharmaceutically acceptable carriers, excipients or diluents. Such compositions may be administered in any convenient or suitable route such as by parenteral (e.g. intraperitoneal, subcutaneous, intraarterial, intravenous, intramuscular, intrathecal, intracerebral, intraocular), oral (including sublingual), nasal, transmucosal or topical routes. In circumstances where it is required that appropriate concentrations of the molecules are delivered directly to the site in the body to be treated, administration may be regional rather than systemic. Regional administration provides the capability of delivering very high local concentrations of the molecules to the required site and thus is suitable for achieving the desired therapeutic or preventative effect whilst avoiding exposure of other organs of the body to the vectors and molecules and thereby potentially reducing side effects.

As will be appreciated by those skilled in the art, the choice of pharmaceutically acceptable carrier or diluent will be dependent on the route of administration and on the nature of the condition and subject to be treated. The particular carrier or diluent and route of administration may be readily determined by a person skilled in the art. The carrier or diluent and route of administration should be carefully selected so as to ensure activity of the zinc delivery agent upon arrival at the site of action.

Examples of pharmaceutically acceptable carriers or diluents are demineralised or distilled water; saline solution; vegetable based oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example, ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrridone; agar; carrageenan; gum tragacanth or gum acacia, and petroleum jelly. Typically, the carrier or carriers will form from 10% to 99.9% by weight of the compositions.

A person skilled in the art will readily be able to determine appropriate formulations for the zinc delivery agent to be administered using conventional approaches. Techniques for formulation and administration may be found in, for example, Remington (1980) Remington's Pharmaceutical Sciences, Mack Publishing Co., Easton, Pa., latest edition; and Niazi (2009) Handbook of Pharmaceutical Manufacturing Formulations, Informa Healthcare, New York, second edition, the entire contents of which are incorporated by reference.

Identification of preferred pH ranges (where appropriate) and suitable excipients is routine in the art, for example, as described in Katdare and Chaubel (2006) Excipient Development for Pharmaceutical, Biotechnology and Drug Delivery Systems (CRC Press).

In some embodiments, the zinc delivery agent may be formulated for oral administration in a dosage form such as a tablet, pill, capsule, liquid, gel, syrup, slurry, suspension, lozenge and the like for oral ingestion by a subject. In particular embodiments, the compound or agent is formulated for oral administration in a solid dosage form, such as a tablet, pill, lozenge or capsule. In such embodiments, the pharmaceutically acceptable carrier may comprise a number of excipients including, but not limited to, a diluent, disintegrant, binder, lubricant, and the like.

Suitable diluents (also referred to as “fillers”) include, but are not limited to, lactose (including lactose monohydrate, spray-dried monohydrate, anhydrous, etc.), mannitol, xylitol, dextrose, sucrose, sorbitol, compressible sugar, isomalt, microcrystalline cellulose, powdered cellulose, starch, pregelatinised starch, dextrates, dextran, dextrin, dextrose, maltodextrin, calcium carbonate, dibasic calcium phosphate, tribasic calcium phosphate, calcium sulfate, magnesium carbonate, magnesium oxide, poloxamers, polyethylene oxide, hydroxypropyl methyl cellulose, silicates (e.g. silicon dioxide), polyvinyl alcohol, talc, and combinations thereof.

Suitable disintegrants include, but are not limited to, sodium carboxymethyl cellulose, pregelatinised starch, calcium carboxymethyl cellulose, croscarmellose sodium, crospovidone, polyvinylpyrrolidone, methylcellulose, sodium starch glycolate, microcrystalline cellulose, lower alkyl-substituted hydroxypropyl cellulose, starch, sodium alginate and combinations thereof. Suitable binders include, but are not limited to, microcrystalline cellulose, gelatine, sugars, polyethylene glycol, natural and synthetic gums, polyvinylpyrrolidone, pregelatinized starch, hydroxypropyl cellulose, hydroxypropyl methylcellulose and combinations thereof. Suitable lubricants include, but are not limited to, magnesium stearate, calcium stearate, zinc stearate, sodium stearyl fumarate, polyethylene glycol and combinations thereof.

Pharmaceutical formulations for parenteral administration include aqueous solutions of a suitable compound or agent in water-soluble form. Additionally, suspensions of the compound or agent may be prepared as appropriate oily injection suspensions. Suitable lipophilic solvents or carriers include fatty oils such as sesame oil, or synthetic fatty acid esters, such as ethyl oleate or triglycerides. Aqueous injection suspensions may contain substances that increase the viscosity of the suspension, such as sodium carboxymethyl cellulose, sorbitol or dextran. Optionally, the suspension may also contain suitable stabilisers or agents that increase the solubility of the compounds to allow for the preparation of highly concentrated solutions.

Sterile solutions may be prepared by combining the zinc complex in the required amount in the appropriate solvent with other excipients as described above as required, followed by sterilization, such as filtration. Generally, dispersions are prepared by incorporating the various sterilised active compounds into a sterile vehicle which contains the basic dispersion medium and the required excipients as described above. Sterile dry powders may be prepared by vacuum- or freeze-drying a sterile solution comprising the active compounds and other required excipients as described above.

The pharmaceutical forms suitable for injectable use include sterile injectable solutions or dispersions and sterile powders for the preparation of sterile injectable solutions. Such forms should be stable under the conditions of manufacture and storage and may be preserved against reduction, oxidation and microbial contamination. For injection, compositions of the invention may be formulated in aqueous solutions, suitably in physiologically compatible buffers such as Hanks' solution, Ringer's solution or physiological saline buffer. For transmucosal administration, penetrants appropriate to the barrier to be permeated are used in the formulation. Such penetrants are generally known in the art.

It will be understood that the specific dose level of the zinc delivery agent for any particular subject will depend upon a variety of factors including, for example, the activity of the agent, the half-life of the agent (or of a complex formed between the zinc ion and the amino acid or salt thereof), the age, body weight, general health and diet of the individual to be treated, the time of administration, rate of excretion, and combination with any other treatment or therapy. Single or multiple administrations can be carried out with dose levels and pattern being selected by the treating physician. A broad range of doses may be applicable. Considering a patient, for example, from about 0.1 mg to about 1 mg of agent may be administered per kilogram of body weight per day. Dosage regimens may be adjusted to provide the optimum therapeutic response. For example, several divided doses may be administered daily, weekly, monthly or other suitable time intervals or the dose may be proportionally reduced as indicated by the exigencies of the situation.

In exemplary embodiments of the present disclosure it is contemplated that the zinc delivery agent may be administered to a subject daily or less than daily, for example every second day or every third day for the duration of treatment required to achieve the desired outcome. Administration may be continuous, for example on a daily basis or every second day, or may be intermittent with spacing between administrations determined by the treating medical professional depending on response of the subject to treatment and progress of the subject during the course of treatment.

The present invention also contemplates combination therapies, wherein the zinc delivery agent as described herein is coadministered with other suitable agents that may facilitate the desired therapeutic or prophylactic outcome. The term “coadministered” mean simultaneous administration in the same formulation or in two different formulations via the same or different routes or sequential administration by the same or different routes. The term “simultaneously” means that the active agents are administered at substantially the same time. The term “sequential” administration means a time difference of from seconds, minutes, hours or days between administration of the agents. Administration may be in any order.

Each embodiment described herein is to be applied mutatis mutandis to each and every embodiment unless specifically stated otherwise.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### Examples

The following examples are illustrative of the disclosure and should not be construed as limiting in any way the general nature of the disclosure of the description throughout this specification.

### General Methods

Data analyses. All data are expressed as mean±SEM, with n being the number of rats/mice/humans or arteries isolated from separate animals unless stated otherwise. Each sigmoidal concentration-response curve was fitted using Prism 8 (GraphPad Software, USA). In some cases, the last data point (i.e. highest concentration) was imputed (replication of the next highest concentration) for better sigmoidal fitting. The pEC₅₀ values (the negative log₁₀M of drug concentration that decreases the response by 50%) and Eₘₐₓ (maximum response) were determined for each tissue and averaged.

Two-tailed Student's paired and unpaired t test were used to analyze the differences between two variables and one-way analysis of variance (1-way ANOVA) with Dunnett's post-test was used to compare means between three or more variables. Repeated measures 1-way ANOVA (mixed-effects if there were missing values) with Dunnett's post-test was used to test concentration-dependent changes from baseline values within the same group. The p values from the post-tests are reported. For in vivo experiments and comparison of two or more concentration-response curves, repeated measures (mixed-effects if there were missing values) two-way analysis of variance (2-way ANOVA) with Sidak's post-test for multiple comparisons of treatment, time or dose were used. The adjusted p values after individual comparisons (post-test) are reported. Values of p<0.05 were considered statistically significant.

For in vivo haemodynamic parameters in anaesthetized rats and mice, the average of the values at 5 min before (−5) and immediately before addition (0 min) of the first dose of the test drug were used as baseline. The values at 2, 3, 4, 5 and 10 min after administration of a single dose of the test drug or vehicle equivalent were measured as well as the change from baseline values in MAP at the 5 min (close to the peak effect) and the area under the curve (AUC) for the change from the baseline values (Prism 8 software).

For in vivo measurements of cutaneous blood flow, an average of 3 s consecutive values calculated every 5 s were computed by LabChart software and the data were transformed as percentage of baseline (an average of 1.5 min recording before each dose). The area under the curve (AUC) for change from baseline values was calculated in Prism 8 software. MAP, hindquarter flow and conductance were taken immediately before and every 5 min after a single dose of treatment.

Functional in vitro protocols in isolated arteries. Human internal mammary artery and saphenous vein or a range of vessels from rats and mice (left main or anterior descending coronary arteries [250-400 μm internal diameter, i.d.], second order pulmonary interlobar arteries [300-600 μm i.d.], second or third order mesenteric arteries [200-350 μm i.d.], interlobar renal arteries [250-350 μm i.d.], saphenous arteries or veins [300-600 μm i.d.]), or the thoracic aorta were dissected and placed in ice-cold PSS-A with the following composition (mmol/L, mM): NaCl 119; KCl 4.69; MgSO₄·7H₂O 1.17; KH₂PO₄ 1.18; glucose 5.5; NaHCO₃ 25; CaCl₂·6H₂O 2.5 saturated with carbogen (O₂ 95%; CO₂ 5%) at pH 7.4. In addition, rat middle cerebral (250-400 μm i.d.) and basilar (250-450 μm i.d.) arteries were used and for these vessels the PSS-A contained 1.5 mM CaCl₂ to minimize the occurrence of spontaneous contractions.

After isolation of vessels, ˜2 mm length segments of arteries were mounted in myograph chambers (Model 610M and 620M; Danish Myo Technology, Denmark) containing PSS-A at 37° C. for isometric force measurement as described previously. Contractile and relaxation responses using rat and mice isolated vessels were recorded with LabChart 7 and a PowerLab 4/30 A/D converter (AD Instruments Pty Ltd, Australia) while those including human isolated vessels were recorded with LabChart 8 (AD Instruments) and Myodaq and Myodata 2.01 (Maastricht University, Maastricht, Netherlands). To normalize the basal conditions, the vessels were passively stretched according to a normalization protocol and adjusted to a diameter setting of 90% of that determined for an equivalent transmural pressure of 100 mmHg (30 mmHg for veins). After allowing the tissues to equilibrate for 30 min, the arteries were exposed to a potassium depolarizing solution (124 mM K⁺ replacing Na⁺ in PSS; termed KPSS) and noradrenaline (10 μM) for 2 min (chemicals, their source and preparation are provided in the online supplement). A second exposure to KPSS solution (only) was used to provide a reference contraction.

In vivo blood pressure and flow measurement. Rats and mice were anaesthetized by inhalation of 5% isoflurane (in O₂) followed by i.p. injection of pentobarbitone in rats (60 mg/kg; TROY Laboratories, NSW, Australia, maintained by top up doses of 10-20 mg/kg i.v.) or i.p. injection of urethane (2.5 g/kg; Sigma, maintained by top up doses of 0.05-0.1 g/kg i.v.). Animals were placed on a homeostatic blanket system to maintain body temperature at 37±1° C. Lignocaine (1% s.c.) was injected for placement of a tracheotomy tube (for mechanical ventilation), carotid artery and jugular vein catheters.

All drug treatments were administered via the jugular vein catheter. Pulsatile blood pressure, mean arterial pressure (MAP) and heart rate (HR; beats/min) were measured via the carotid artery catheter filled with heparinized (10 U/ml; Pfizer, NY, USA) saline 0.9% and connected to a pressure transducer (Argon Medical Devices, Athens, Greece) and the output was recorded with LabChart 7 and a Powerlab 8SP (AD Instruments). A Silastic-cuffed Doppler flow probe placed around the aorta just above the bifurcation was used for constant recording of hindquarter blood flow (kHz) and derivation of hindquarter vascular conductance (kHz/MAP mmHg).

### Example 1—Zinc Delivery Agents Cause Vasorelaxation of Rat Mesenteric Arteries

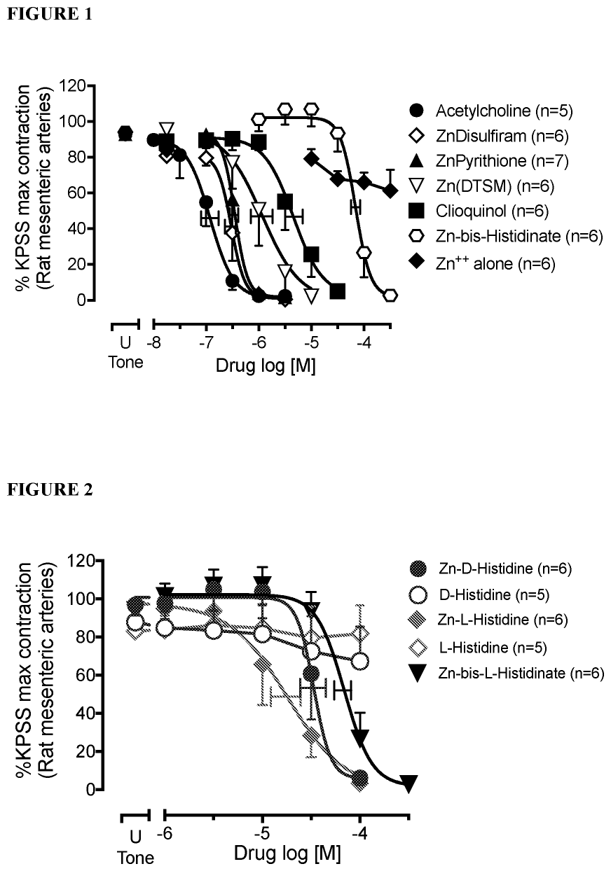

Five structurally dissimilar zinc delivery agents (zinc pyrithione, zinc disulfiram, clioquinol, Zn(DTSM) and zinc bis(histidinate)) were administered to rat mesenteric arteries according to the protocols above and the extent to which the arteries contracted were recorded. Acetylcholine and clioquinol were also administered and contractions recorded. The administration of the zinc complexes showed relaxation of rat mesenteric arteries (see FIG. 1) that were contracted with a thromboxane mimetic (U46619).

As shown in FIG. 1, the administration of a zinc delivery agent caused a concentration-dependent relaxation of rat mesenteric arteries. In the absence of a zinc delivery agent, administration of extracellular zinc alone had minimal effects, indicating that the vasorelaxation was attributable to an increase in the concentration of intracellular zinc.

FIG. 2 compares the relaxation of rat mesenteric arteries upon administration of either zinc histidinate (i.e. zinc and histidine in a 1:1 ratio), zinc bis(histidinate) (i.e. zinc and histidine in a 1:2 ratio) or histidine alone, where histidine is present in the D- or L-form. Administration of zinc and histidine in a 1:2 stoichiometric ratio resulted in greater relaxation, when compared to zinc and histidine present in a 1:1 ratio. Administration of histidine alone did not provide the same relaxation.

### Example 2—Zinc Bis(Histidinate) Causes an Increase in Cutaneous Blood Flow

Rats were anaesthetized with urethane (2 g/kg i.p.) after light anaesthesia with inhalation of 5% isoflurane. A laser Doppler probe (OxyFlo probe MSP300XP; ADI Blood Flowmeter) was placed on one hind paw (hair removed with depilatory cream). The animal was allowed to stabilize for 30 min to establish baseline parameters. A single i.v. dose of zinc bis(histidinate) was then administered at 3 mg/kg. The haemodynamic and cutaneous blood flow measurements by laser Doppler flow (LDF) were recorded continuously for 15 min. Then, the effects of 10 mg/kg and 30 mg/kg doses of zinc bis(histidinate) were tested.

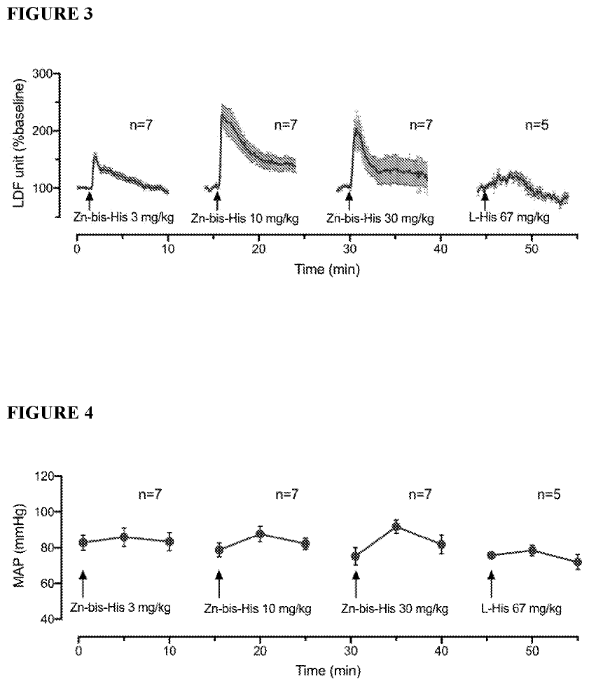

FIG. 3 shows the level of cutaneous blood flow in the hind paw of a rat after administration of either zinc bis(histidine) or L-histidine. Administration of zinc bis(histidine) resulted in increased blood flow, whereas the administration of L-histidine alone did result in the same increase.

### Example 3—Zinc Bis(Histidinate)-Mediated Increase in Cutaneous Blood Flow is not Accompanied by a Decrease in Arterial Blood Pressure

An increase in cutaneous blood flow associated with vasorelaxation typically results in a decrease in arterial pressure. Mean arterial pressure in a rat after administration of zinc bis(histidine) (3 mg/kg, 10 mg/kg or 30 mg/kg) or L-histidine (67 mg/kg) was measured by a pressure transducer attached to a catheter inserted in carotid artery as described in Example 2.

FIG. 4 shows the mean arterial pressure of a rat after administration of zinc bis(histidine) at various concentrations or a dose-equivalent amount of L-histidine alone. Arterial pressure was measured for 10 minutes by carotid artery catheter attached to a pressure transducer. Although zinc bis(histidinate) induces local vasorelaxation and increases cutaneous blood flow, the expected decrease in arterial pressure was not observed.

### Example 4—Zinc Bis(Histidinate)-Mediated Increase in Cutaneous Blood Flow is Dependent on Sensory Nerve Activity

In order to better understand the effect of zinc bis(histidinate) on cutaneous blood flow, vasodilation mediated by sensory nerves was blocked by pre-treatment of the rat with a CGRP (calcitonin gene-related peptide) receptor antagonist. A dose of 3 mg/kg BIBN4096 (olcegepant, a known CGRP receptor antagonist was administered intravenously by slow infusion over 10 minutes prior to subsequent administration of zinc bis(histidinate) and measurement of cutaneous blood flow by LDF as described in Example 2.

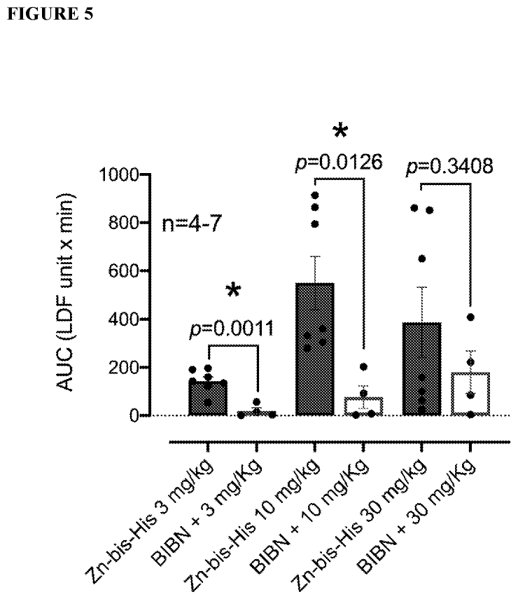

FIG. 5 shows the area under the vasodilation curve obtained by LDF of a rat hind paw. Pre-treatment of the paw with BIBN4096 to block vasodilation attributed to sensory nerves resulted in a reduction in vasodilation induced by administration of zinc bis(histidinate). This shows that an increase in cutaneous blood flow is dependent on sensory nerve activity, since the inhibition of CGRP receptors with BIBN4096 prior to administration of zinc bis(histidinate) reduced cutaneous blood flow.

### Example 5—Zinc Bis(Histidinate) Improves Renal Blood Flow, Vascular Resistance and Renal Oxygen Delivery During Cardiopulmonary Bypass Surgery

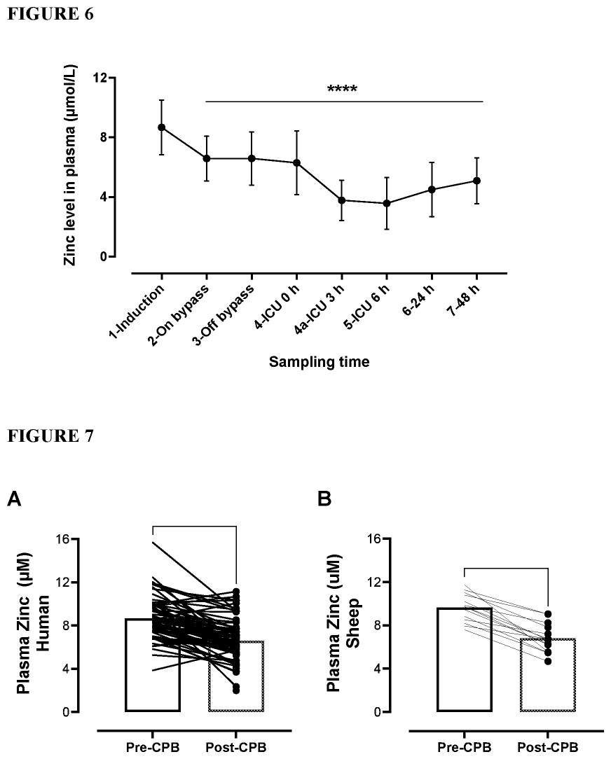

In a cohort of 82 patients undergoing cardiopulmonary bypass, the inventors have shown that circulating zinc levels, as determined using inductively coupled plasma mass spectroscopy (ICP-MS), are significantly depleted during cardiopulmonary bypass surgery and further decreased during recovery in the intensive care unit (FIG. 6). The inventors then replicated these findings in a large animal (ovine) model of cardiopulmonary bypass, showing a significantly decreased plasma zinc level after 2 h cardiopulmonary bypass surgery (FIG. 7).

Therefore the inventors proposed that restoring zinc could be a therapeutic approach to rescue brain and kidney injury resulting from cardiopulmonary bypass. The effect of intra-operative administration of zinc bis(histidinate) was observed during cardiopulmonary bypass surgery in sheep. All experiments were approved by the Animal Ethics Committee of the Florey Institute of Neuroscience and Mental Health under guidelines of the National Health and Medical Research Council of Australia.

Two merino ewes (35-50 kg bodyweight) were housed in individual metabolic cages with access to 5 litres of water and 800 g of oaten chaff daily. The sheep underwent a preparatory surgical procedure under isoflurane anaesthesia for the implantation of a transit-time flow probe around the renal artery for measurement of renal blood flow. Fibre-optic probes were inserted into the cerebral cortex, renal cortex and renal medulla for measurement of tissue perfusion, pO₂ and temperature (see Calzavacca et al., 2015, Am J Physiol Regul Integr Comp Physiol 308, R832-839). Catheters were inserted into the renal and jugular veins, carotid artery and bladder. The experiments were undertaken five days later. Intra-operative analgesics and antibiotics were administered as described previously (Lankadeva et al., 2019, Kidney Int 95 1338-1346; Evans et al., 2020, Am J Physiol Regul Integr Comp Physiol 318, R206-R213; Lankadeva et al., 2021, Acta Physiologica doi.org/10.1111/apha.13596, e13596). Mean arterial pressure, renal blood flow, cerebral cortical, renal cortical and renal medullary tissue perfusion, and temperature were continuously recorded across a series of experimental periods. Arterial, mixed venous and renal venous blood samples were collected every 30 min for blood oximetry and biochemistry (ABL systems 625, Copenhagen, Denmark).

During the first 30 minutes, sheep were awake and unrestrained in their home metabolic cage. Anaesthesia was induced with sodium thiopental (15 mg/kg) and after endotracheal intubation, maintained on isoflurane (2.0%-2.5%) via an artificial ventilator prior to cardiopulmonary bypass or via a membrane oxygenator during cardiopulmonary bypass (FiO2: 60%). This FiO2 was chosen because it is standard during cardiac surgery at the inventors affiliated clinical centre. From the onset of anaesthesia, sheep received a maintenance infusion of compound sodium lactate (2 mL/kg/h; Baxter, NSW, Australia) for the entire duration of the experiment. Once stable anaesthesia was attained, a second experimental period (30 minutes) commenced. Cardiopulmonary bypass was then achieved at a target pump flow of 80 mL/kg/min, a target MAP of 65 mmHg, and a target body temperature of 34.5° C. The prefusion circuit was primed with 300-500 mL of blood from a donor animal, 1 g cefazolin (AFT Pharmaceuticals, NSW, Australia), 50 mL mannitol (20% wt/vol Osmitrol, Baxter, NSW, Australia) and 10 000 IU heparin, made up to 1.3 L with compound sodium lactate. Metaraminol (Metaraminol Montrose, Montrose Life Sciences, NSW, Australia) was administered in boluses of ˜0.25 mg (using a 0.5 mg/mL solution) only in cases where increasing pump flow was not sufficient to attain the target mean arterial pressure during cardiopulmonary bypass. Once stable cardiopulmonary bypass was attained, the sheep was maintained for ˜2 hours before starting a zinc bis(histidinate) infusion (18 mg/ml solution made freshly in 0.45% saline solution and filtered). For the first 20 min, volume equivalent to 3 mg/kg zinc bis(histidinate) was infused, followed by 20 minute of 10 mg/kg and then 20 min of 30 mg/kg zinc bis(histidinate). Plasma was collected every 30 min following the start of anaesthesia. Zinc levels in plasma were analysed using inductively coupled plasma mass spectroscopy (ICP-MS).

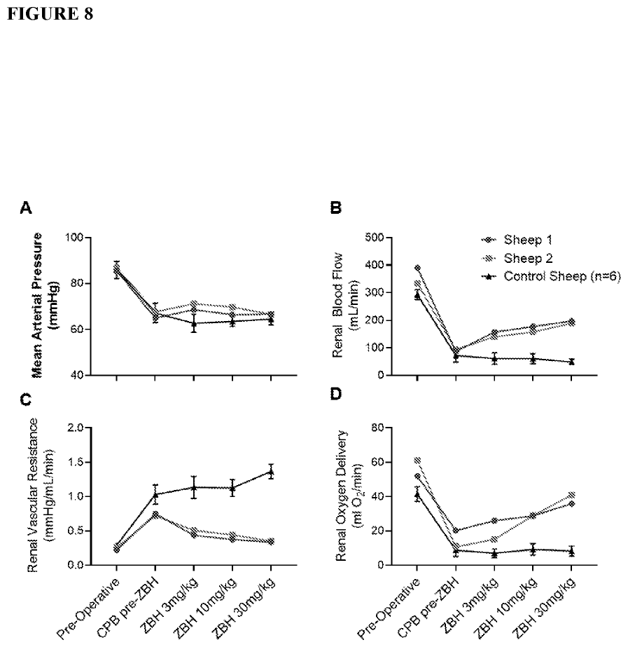

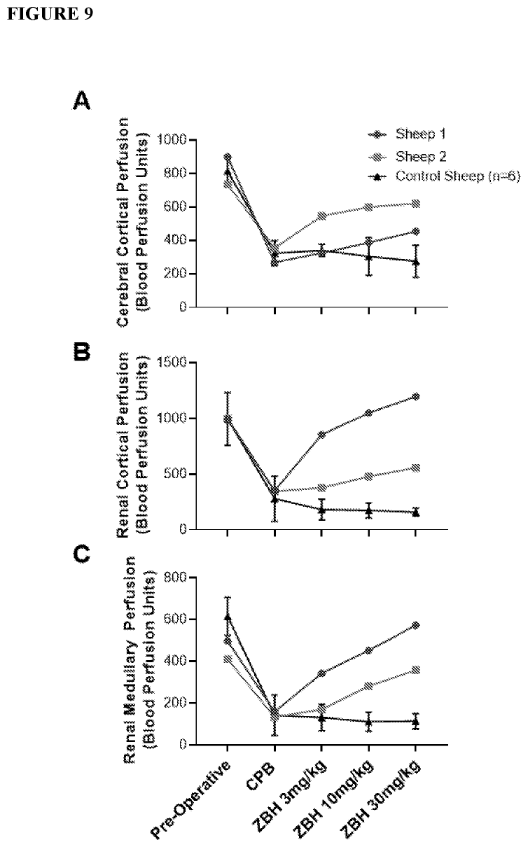

After 2 h cardiopulmonary bypass surgery, there was a decrease in renal blood flow (FIG. 8B) and increased vascular resistance (FIG. 8C) leading to decreased renal oxygen delivery (FIG. 8C), although the systemic mean arterial pressure was maintained at the clinically acceptable target of 65 mmHg (FIG. 8A). The decrease in renal blood flow was also accompanied by decreased cerebral cortical (FIG. 9A), renal cortical (FIG. 9B) and renal medullary (FIG. 9C).

Thus, when the intracellular zinc levels were elevated using i.v. zinc bis(histidinate), there was a dose-dependent improvement in renal blood flow, vascular resistance and renal oxygen delivery (FIG. 8). Correspondingly, cerebral cortical, renal cortical and renal medullary microcirculatory perfusion was rescued in ovine cardiopulmonary bypass (FIG. 9), and importantly without compromising systemic mean arterial pressure (FIG. 8A). The guiding principle for target systemic mean arterial pressure during human cardiopulmonary bypass is that cerebral blood flow autoregulation must remain functional (Murkin et al., 1987, Anesth Analg 66, 825-832; Wahba et al., 2020, Eur J Cardiothorac Surg 57, 210-251). A target mean arterial pressure of >50 mmHg during human cardiopulmonary bypass is currently considered acceptable for avoidance of compromised cerebral blood flow. Thus, our target systemic mean arterial pressure of 65 mmHg during ovine cardiopulmonary bypass was similar to that deployed in the inventors' associated clinical centre in human cardiopulmonary bypass. However, the inventors suggest that the currently accepted clinical mean arterial pressure targets for cardiopulmonary bypass are suboptimal for maintaining adequate cerebral microcirculatory perfusion during the surgical procedure. Metaraminol is a vasopressor commonly used during human cardiopulmonary bypass to maintain systemic mean arterial pressure. Metaraminol appears to chiefly induce vasoconstriction in the extrarenal vasculature, as the inventors have previously found it increased systemic vascular conductance but not renal vascular resistance in sheep during cardiopulmonary bypass. The findings described herein that zinc bis(histidinate) does not compromise systemic mean arterial pressure is significant, alleviating any increased requirement for vasopressors during cardiopulmonary bypass.

1. A method for preventing or minimising hypoperfusion in a subject during surgery, the method comprising administering to the subject a therapeutically effective amount of a zinc delivery agent, wherein the zinc delivery agent comprises a zinc ion and at least one amino acid or a salt thereof.

2. A method according to claim 1, wherein the administration of the zinc delivery agent results in an improvement in one or more of cerebral cortical perfusion, renal cortical perfusion and renal medullary perfusion during the surgery.

3. A method according to claim 1, wherein the administration of the zinc delivery agent results in an improvement in one or more of renal blood flow, vascular resistance and renal oxygen delivery during the surgery.

4. (canceled)

5. A method according to claim 1, wherein the administration of the zinc delivery agent maintains a systemic mean arterial pressure of at least about 50 mmHg.

6. A method according to claim 1, wherein the administration of the zinc delivery agent maintains a systemic mean arterial pressure of at least about 60 mmHg.

7. A method according to claim 1, wherein the zinc delivery agent is administered prior to surgery, during surgery and/or after surgery.

8. A method according to claim 1, wherein the zinc delivery agent is administered intravenously.

9. A method according to claim 1, wherein the surgery is vascular surgery, abdominal surgery, orthopaedic surgery or transplant surgery.

10. A method according to claim 1, wherein the surgery is cardiac surgery requiring cardiopulmonary bypass.

11. A method according to claim 1, wherein the method prevents, or reduces the risk of, the subject suffering from a post-operative complications associated with reduced blood flow and/or reduced oxygen delivery to tissues.

12. A method according to claim 11, wherein the post-operative complication is delirium, acute kidney injury or chronic kidney disease.

13. A method according to claim 11, wherein the post-operative complication is associated with hypoperfusion and/or hypoxia in a subject.

14. A method for the treatment or prevention of a vascular disease or disorder in a subject, the method comprising administering to the subject a therapeutically effective amount of a zinc delivery agent, wherein the zinc delivery agent comprises a zinc ion and at least one amino acid or a salt thereof.

15. A method according to claim 14, wherein the vascular disease or disorder is primary Raynaud's phenomenon or secondary Raynaud's phenomenon.

16. (canceled)

17. A method for the treatment or prevention of an autoimmune disease associated with Raynaud's phenomenon, the method comprising administering to a subject in need thereof a therapeutically effective amount of a zinc delivery agent, wherein the zinc delivery agent comprises a zinc ion and at least one amino acid or a salt thereof; wherein the autoimmune disease associated with Raynaud's phenomenon is systemic sclerosis, a mixed connective tissue disease, systemic lupus erythematosus, primary Sjogren's syndrome, a myositis-spectrum disorder or fibromyalgia syndrome.

18. (canceled)

19. (canceled)

20. A method according to claim 1, wherein the administration of the zinc delivery agent results in vasodilation.

21. (canceled)

22. A method according to claim 2, wherein the blood vessels are cutaneous or renal blood vessels.

23. (canceled)

24. A method according to claim 1, wherein the zinc ion and the at least one amino acid, or salt thereof, are provided in the form of a complex, or form a complex in vivo after administration to the subject.

25. A method according to claim 1, wherein the amino acid or salt thereof is histidine or a histidine salt.

26. A method according to claim 1, wherein the delivery agent comprises zinc bis(histidinate).
